(19) United States
(12) Patent Application Publication
(10) Pub. No.: US 2025/0138637 A1
(43) Pub. Date: May 1, 2025

(54) WEARABLE DEVICE, SIGNAL PROCESSING METHOD AND WEARABLE SYSTEM

(71) Applicant: GOERTEK INC., Weifang (CN)

(72) Inventors: Huajie SAN, Weifang (CN); Lin Wang, Weifang (CN)

(21) Appl. No.: 19/009,167

(22) Filed: Jan. 3, 2025

Related U.S. Application Data
(63) Continuation of application No. PCT/CN2023/106290, filed on Jul. 7, 2023.

(30) Foreign Application Priority Data
Jul. 28, 2022 (CN) 202210903464.8

(51) Int. Cl. G06F 3/01

(52) U.S. Cl. CPC G06F 3/015

(57) ABSTRACT

Disclosed are a wearable device, a signal processing method and a wearable system, the wearable device includes a receiving electrode and a control circuit of the wearable device. The receiving electrode can receive an excitation signal sent by a transmitting wearable device through the skin of the user. The control circuit of the wearable device is used to obtain the present skin impedance of the user through the receiving electrode and adjust the gain of the excitation signal; and when the excitation signal is received, the excitation signal is amplified based on the adjusted gain to determine the present click position of the user.

### CROSS-REFERENCE TO RELATED APPLICATIONS

This application is a continuation application of International Application No. PCT/CN2023/106290, filed on Jul. 7, 2023, which claims priority to Chinese Patent Application No. 202210903464.8, filed on Jul. 28, 2022. The disclosures of the above-mentioned applications are incorporated herein by reference in their entireties.

### TECHNICAL FIELD

The present application relates to the technical field of augmented reality/virtual reality (AR/VR), and in particular to a wearable device, a signal processing method, and a wearable system.

### BACKGROUND

Electromyography (EMG) technology is now widely used in the augmented reality/virtual reality (AR/VR) field, primarily for hand motion recognition and AR/VR applications to enable specific touch and selection functions, such as clicking or menu selection. These functions are achieved by collecting human excitation signals through electrodes that contact the skin to perform motion analysis. However, since human skin resistance varies with physical conditions or environmental factors—such as dryness, sweating, or moisture—the skin resistance and the collected excitation signals will fluctuate. If these variations are not addressed, the recognition results will differ, leading to a reduced recognition success rate.

### SUMMARY

The main objective of the present application is to provide a wearable device, a signal processing method and a wearable system, aiming to improve the success rate of recognition results.

In order to achieve the above objective, an embodiment of the present application provides a wearable device, including: a receiving electrode; and a control circuit of the wearable device electrically connected to the receiving electrode.

When the wearable device is worn by a user, the receiving electrode is configured to contact skin of the user and receive an excitation signal sent by a transmitting wearable device through the skin of the user.

The wearable device is configured to receive the excitation signal through the receiving electrode.

The control circuit of the wearable device is configured to:
obtain, by the receiving electrode, a present skin impedance of the user, a functional relationship between a skin impedance of the user and a gain of the excitation signal;
adjust, based on the present skin impedance and the functional relationship between the skin impedance of the user and the gain of the excitation signal, the gain of the excitation signal; and
amplify the excitation signal based on the adjusted gain to determine a present click position of the user in response to receiving the excitation signal.

An embodiment of the present application further provides a signal processing method, applied to the above wearable device.

In an embodiment, the wearable device includes a receiving electrode.

In an embodiment, the signal processing method includes:
obtaining, from the receiving electrode, a present skin impedance of a user;
obtaining a functional relationship between the skin impedance of the user and a gain of an excitation signal;
adjusting, based on the functional relationship between the skin impedance of the user and the gain of the excitation signal, the gain of the excitation signal; and
amplifying the excitation signal based on the adjusted gain to determine a present click position of the user in response to receiving the excitation signal.

An embodiment of the present application further provides a wearable system, including: a host; and the above wearable device. The wearable device is electrically or wirelessly connected to the host. The control circuit of the wearable device is configured to output, based on the click position, a corresponding click position signal to the host to cause the host to generate a corresponding image and/or audio based on the click position signal.

The present application detects the present skin impedance of the user through the control circuit of the wearable device and determines the actual change in the skin impedance of the user based on the present skin impedance and the initial skin impedance. Based on the actual change in the skin impedance of the user, the gain of the received excitation signal in the control circuit of the wearable device is adjusted accordingly, and the excitation signal is amplified based on the adjusted gain. This ensures that the difference between the amplitude of the excitation signal output by the wearable device and the amplitude of the excitation signal output under normal skin conditions does not exceed a preset amplitude range, thereby avoiding recognition errors caused by the output excitation signal amplitude being too large or too small, and improving the accuracy of click position recognition.

### BRIEF DESCRIPTION OF THE DRAWINGS

In order to more clearly illustrate the technical solutions in the embodiments of the present application or in the related art, drawings used in the embodiments or in the related art will be briefly described below. Obviously, the drawings in the following description are only some embodiments of the present application. It will be apparent to those skilled in the art that other figures can be obtained based on the structures shown in the drawings without creative work.

FIG. 1A is a schematic structural diagram of a wearable device according to an embodiment of the present application.

FIG. 1B is a schematic structural diagram of a wearable device according to an embodiment of the present application.

FIG. 2A is an original waveform of the excitation signal when A, B, and C are clicked in a normal skin condition according to an embodiment of the present application.

FIG. 2B is a waveform for extracting the characteristic values of the excitation signal under the normal skin condition according to an embodiment of the present application.

FIG. 3A is a waveform of the excitation signal when A, B, and C are clicked under a moist skin condition according to an embodiment of the present application.

FIG. 3B is a waveform for extracting the characteristic values of the excitation signal under the moist skin condition according to an embodiment of the present application.

FIG. 4 is a schematic structural diagram of the wearable device according to an embodiment of the present application.

FIG. 5 is a schematic structural diagram of a control circuit of the wearable device according to an embodiment of the present application.

FIG. 6 is a schematic structural diagram of an impedance detection circuit according to an embodiment of the present application.

FIG. 7 is a schematic structural diagram of a switch circuit according to an embodiment of the present application.

FIG. 8 is a schematic flowchart of a signal processing method according to an embodiment of the present application.

FIG. 9 is a schematic flowchart of the signal processing method according to an embodiment of the present application.

FIG. 10 is a schematic structural diagram of a wearable system according to an embodiment of the present application.

The realization of the purpose, functional features and advantages of the present application will be further described with reference to the embodiments and the accompanying drawings.

### DETAILED DESCRIPTION OF THE EMBODIMENTS

Exemplary embodiments of the present application will be described in more detail below with reference to the accompanying drawings. Although exemplary embodiments of the present application are shown in the drawings, it should be understood that the disclosure may be embodied in various forms and should not be limited to the embodiments set forth herein. Rather, these embodiments are provided to provide a thorough understanding of the disclosure, and to fully convey the scope of the disclosure to those skilled in the art.

It should be noted that all of the directional instructions in the embodiments of the present application (such as, up, down, left, right, front, rear . . . ) are only used to explain the relative position relationship and movement of each component under a specific attitude (as shown in the drawings), if the specific attitude changes, the directional instructions will change correspondingly.

Besides, the descriptions in the present application that refer to “first,” “second,” etc. are only for descriptive purposes and are not to be interpreted as indicating or implying relative importance or to implicitly indicate the number of technical features indicated. Thus, a feature defined as “first” or “second” may explicitly or implicitly include at least one of the feature. In addition, technical solutions between the embodiments can be combined with each other, but must be based on the realization of the technical solutions by those skilled in the art, and when the technical solutions are contradictory to each other or cannot be realized, the technical solutions should be considered that the combination does not exist, and the technical solutions are not fallen within the protection scope claimed in the present application.

The human body is a conductor, the body resistance is mainly the skin resistance, and its value is related to factors such as contact voltage, contact area, contact pressure, and skin surface conditions (dryness, tissue damage, sweating, conductive dust, thickness of skin surface keratin), etc. In general, the body resistance can be considered as 1000-2000Ω. Electromyography (EMG) technology mainly collects electrical signals from the surface of the human skin for motion recognition. Changes in body resistance will bring about changes in electrical signals, which in turn affects signal recognition.

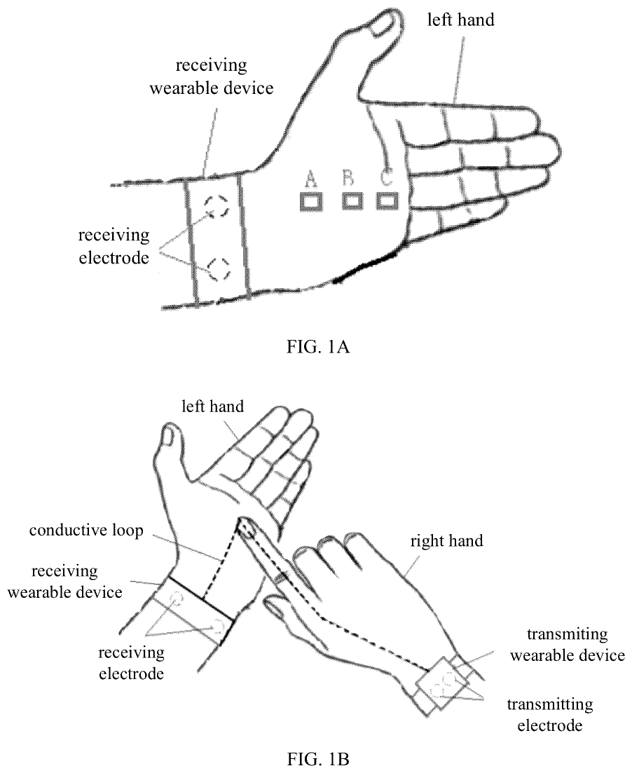

To solve the above problems, as shown in FIG. 1A to FIG. 4, the present application provides a wearable device. The wearable device includes: a receiving electrode 10, a control circuit of the wearable device 20. When the wearable device is worn by a user, the receiving electrode 10 can contact the skin of the user and receive the excitation signal sent by the transmitting wearable device through the skin of the user.

The control circuit of the wearable device 20 is electrically connected to the receiving electrode 10 to receive the excitation signal through the receiving electrode 10.

The control circuit of the wearable device 20 is used to obtain the present skin impedance of the user by the receiving electrode 10, and obtain the functional relationship between the skin impedance of the user and the gain of the excitation signal, so as to adjust the gain of the excitation signal based on the present skin impedance of the user and the functional relationship between the skin impedance of the user and the gain of the excitation signal.

When the excitation signal is received, the excitation signal is amplified by the wearable device 20 based on the adjusted gain to determine the present click position of the user.

In this embodiment, the transmitting wearable device has a transmitting electrode that contacts the skin of the user when the transmitting wearable device is worn on the user. The excitation signal is loaded onto the skin surface through the transmitting electrode and is conducted to the receiving electrode 10 of the wearable device through the human skin. The receiving electrode 10 receives the excitation signal on the skin surface, and after amplification, analog-to-digital conversion, and other processing by the control circuit of the wearable device 20, the analog excitation signal is converted into a digital signal that can be recognized by the machine, and is transmitted to the upper-level device. The voltage value of the converted digital signal is compared with a plurality of position standard characteristic values representing different click positions for matching, and the position corresponding to the position standard characteristic value that best matches the excitation signal collected by the receiving electrode 10 is determined as the present click position of the user based on the matching result.

When a click operation is required, the human body part wearing the transmitting wearable device performs a click action on the human body part wearing the wearable device. The waveform change rules of the excitation signal collected by the receiving electrode 10 at different positions are basically the same, and all of them show click actions, so it is difficult to distinguish different click positions by action recognition alone. When clicking different positions, the resistance between the receiving electrode 10 and the transmitting wearable device is different, the amplitude of the excitation signal waveform collected at the receiving end is different, and the amplitude of the excitation signal output to the upper-level device after amplification, analog-to-digital conversion, etc. by the control circuit of the wearable device 20 is also different. Therefore, the present click position can be determined by comparing and matching the amplitude of the excitation signal output by the wearable device with multiple position standard characteristic values representing different click positions. The transmitting wearable device and/or the wearable device can be a watch, a bracelet or a ring, etc., which is not limited here. This embodiment is illustrated by taking a watch as an example.

For example, in an augmented reality/virtual reality (AR/VR) application, a selection menu appears on a certain part of the hand wearing the wearable device (the left palm is taken as an example here), and the right hand wearing the transmitting wearable device can click on the menu to select and perform other operations. The left palm can be divided into click areas representing different functional modules. In this embodiment, three areas (A, B and C) are defined, as shown in FIG. 1A.

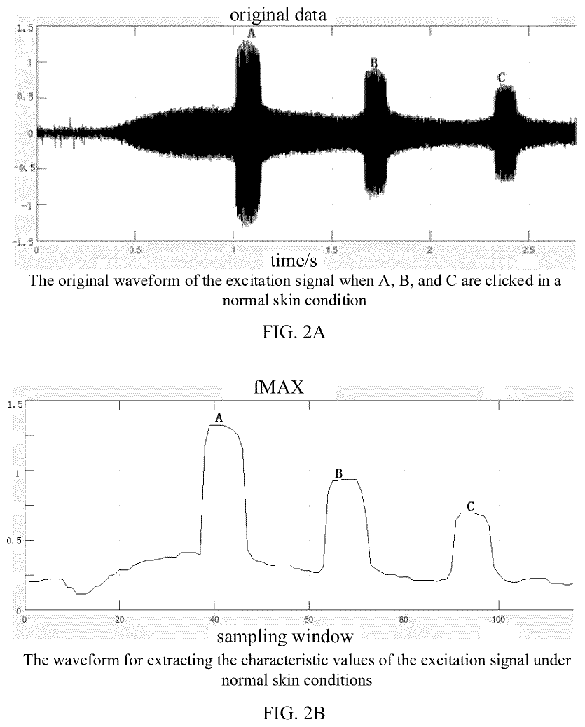

When the left and right hands are in contact, a conductive loop is formed between the transmitting electrode and the receiving electrode 10, as shown by the dotted line in FIG. 1B. When the right hand clicks on the three positions A, B, and C respectively, the resistance of the conductive loop is different due to the different path lengths. When the excitation voltage is fixed, the longer the path, the greater the resistance, the smaller the amplitude of the excitation signal received by the receiving electrode 10, and the smaller the amplitude of the excitation signal output after amplification, analog-to-digital conversion, etc. The shorter the path, the smaller the resistance, the larger the amplitude of the excitation signal received by the corresponding receiving electrode 10, and the larger the amplitude of the excitation signal output after amplification, analog-to-digital conversion, etc. In this embodiment, the resistance between the transmitting electrode-A-receiving electrode 10 is defined as RA1, the resistance between the transmitting electrode-B-receiving electrode 10 is defined as RB1, and the resistance between the transmitting electrode-C-receiving electrode 10 is defined as RC1, RA1<RB1<RC1, and the corresponding collected excitation signal amplitude VA1>VB1>VC1, and the waveforms are shown in FIG. 2A and FIG. 2B. FIG. 2A and FIG. 2B show the waveforms of the excitation signal amplitude collected by clicking on positions A, B, and C respectively under normal skin conditions at room temperature when the excitation voltage, excitation frequency, and skin resistance remain unchanged. At this time, the present skin impedance of the user obtained by the receiving electrode 10 is 3.2 MΩ.

However, human skin resistance will change with the skin surface. For example, when the skin surface sweats and becomes moist, the skin resistance decreases. When the skin resistance changes, the amplitudes VA1, VB1, and VC1 of the excitation signals collected by the receiving electrode 10 will also change.

The relationship between the amplitude of the excitation signal output by the wearable device, the skin impedance, and the gain of the excitation signal by the control circuit of the wearable device 20 is:

$$V\_emg = ( V\_excitation \_voltage / Z ) ⋆ R\_receiving \_circuit ⋆ Gain ,$$

V_emg is the amplitude of the excitation signal output by the wearable device, that is, VA1, VA2, VA3;
V_excitation_voltage is the excitation voltage loaded by the transmitting wearable
device and is a fixed value;
Z is the skin impedance between the transmitting electrode and the receiving electrode 10, i.e., RA1, RB1, RC1;
R_receiving_circuit is the resistance value of the receiving circuit part of the wearable device, which can be regarded as a fixed value; and
Gain is the gain of the control circuit of the wearable device 20 to the excitation signal.

When the V_excitation_voltage, R_receiving_circuit and the gain Gain of the excitation signal of the control circuit of the wearable device 20 remain unchanged, the change in the amplitude V_emg of the excitation signal output by the wearable device is inversely linearly related to the change in the skin impedance Z, that is, the smaller the skin impedance, the larger the amplitude of the excitation signal output by the wearable device; and the larger the skin impedance, the smaller the amplitude of the excitation signal output by the wearable device to the upper-level device.

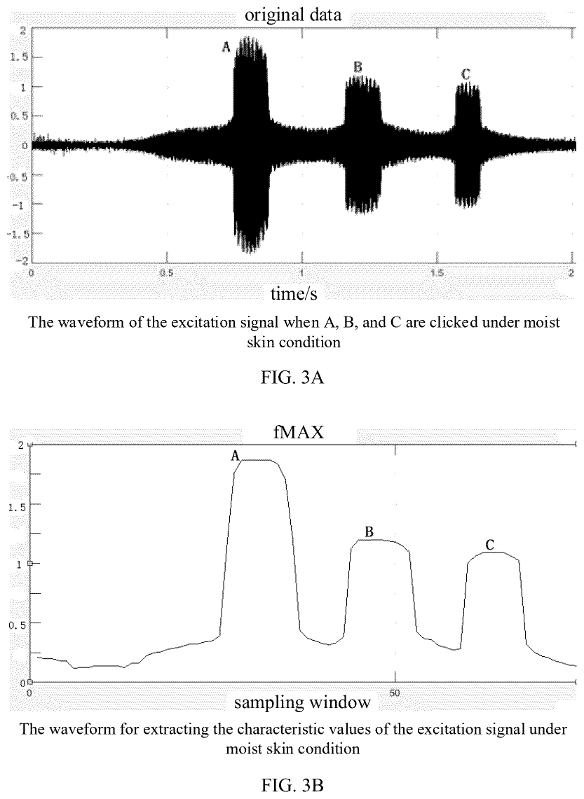

FIG. 3A and FIG. 3B show the waveforms of the amplitude of the excitation signal collected by clicking on the three positions A, B, and C respectively when the excitation voltage and gain remain unchanged and the skin surface is wet. At this time, the present skin impedance of the user obtained by the receiving electrode 10 is 2.2 MΩ. Comparing FIG. 2 and FIG. 3, it can be seen that the skin impedance decreases, and the amplitude of the corresponding excitation signal increases. When the excitation signal transmitted to the upper-level device is compared and matched with multiple position standard signals representing different click positions, the position standard signal representing the click position may be matched with a position standard signal different from the actual click position of the user, which leads to misjudgment.

In order to ensure the accuracy of click position recognition, it is necessary to minimize the difference between the amplitude of the excitation signal output by the wearable device when the skin impedance changes and the amplitude of the excitation signal output by the wearable device under normal skin conditions. The present application measures the present skin impedance of the user through the control circuit of the wearable device 20 to obtain the change relationship of the skin impedance. The gain of the excitation signal is adjusted based on the change rule of the skin impedance and the functional relationship between the skin impedance of the user and the gain of the excitation signal, so that the amplitude of the excitation signal output by the wearable device to the upper-level device follows the change of human skin impedance. Regardless of how the skin impedance of the user changes, it is ensured that the difference between the amplitude of the excitation signal output by the wearable device and the amplitude of the excitation signal output under normal skin conditions does not exceed the preset amplitude range.

In an embodiment, the quantity of receiving electrodes 10 is at least two, and the control circuit of the wearable device 20 measures the human skin impedance between the two receiving electrodes 10 to obtain the present skin impedance of the user. The human body is a conductor, and a small piece of skin changes in the same way as the entire human skin. Therefore, the impedance change rule of this small piece of skin can represent the impedance change rule of the entire arm skin.

Based on the functional relationship between the present skin impedance of the user and the skin impedance of the user and the gain of the excitation signal, V_emg=(V_excitation_voltage/Z)*R_receiving_circuit*Gain, it can be known that when the V_excitation_voltage and R_receiving_circuit remain unchanged, when the skin impedance Z between the transmitting electrode and the receiving electrode 10 changes, it is necessary to ensure that the amplitude difference between the excitation signal output by the wearable device and the amplitude difference of the excitation signal output under the normal skin state does not exceed the preset amplitude range. Then, when the human body impedance changes, the gain Gain of the control circuit of the wearable device 20 to the excitation signal needs to be adjusted accordingly. The skin impedance Z between the transmitting electrode and the receiving electrode 10 is in an inverse linear relationship with the gain Gain of the control circuit of the wearable device 20 to the excitation signal. When the skin impedance Z becomes larger, the gain Gain of the control circuit of the wearable device 20 to the excitation signal needs to be reduced accordingly. When the skin impedance Z becomes smaller, the gain Gain of the control circuit of the wearable device 20 to the excitation signal needs to be increased accordingly. In an embodiment, based on the functional relationship between the present skin impedance of the user and the skin impedance of the user and the gain of the excitation signal, the target gain can be determined as:

G1=G0*Z1/Z0; where G1 is the target gain, G0 is the initial gain, Z1 is the present skin impedance, and Z0 is the initial skin impedance.

The control circuit of the wearable device 20 adjusts the gain of the received excitation signal to the target gain, so as to adjust the excitation signal output to the upper-level device in accordance with the change of skin impedance when the skin impedance changes, so that the difference between the excitation signal output to the upper-level device after the skin impedance changes and the excitation signal output to the upper-level device under normal skin conditions is within the preset amplitude range, ensuring that the upper-level device can correctly identify the present click position of the user. This embodiment is applied in the AR/VR field, and can replace the handle to free both hands and realize true bare-hand interaction. The menu selection function in the bare-hand interaction determines the click position by determining the different resistances of the circuit composed of the transmitting electrode, human skin and receiving electrode 10 when clicking at different positions, thereby selecting the corresponding menu function. Based on the recognition of click action, this embodiment realizes the function of click position recognition, making its function more complete.

In this embodiment, the initial skin impedance can be set before leaving the factory. Or, the skin impedance of the user when wearing it for the first time can be collected as the initial skin impedance. Since each person's skin condition is different, different users set the initial skin impedance and position standard characteristic values based on their own skin conditions when wearing it. The initial value is collected when the user wears it for the first time. If the user is not changed later, there is no need to collect it again. If the user is changed, the initial value needs to be collected again.

The present application detects the present skin impedance of the user through the control circuit of the wearable device, and determines the actual change of the skin impedance of the user based on the present skin impedance and the initial skin impedance of the user. based on the actual change of the skin impedance of the user, the gain of the received excitation signal of the control circuit of the wearable device is adjusted accordingly, and the excitation signal is amplified based on the adjusted gain, so that the difference between the amplitude of the excitation signal output by the wearable device and the amplitude of the excitation signal output under normal skin conditions does not exceed the preset amplitude range, avoiding the error recognition caused by the amplitude of the output excitation signal being too large or too small, and improving the accuracy of click position recognition.

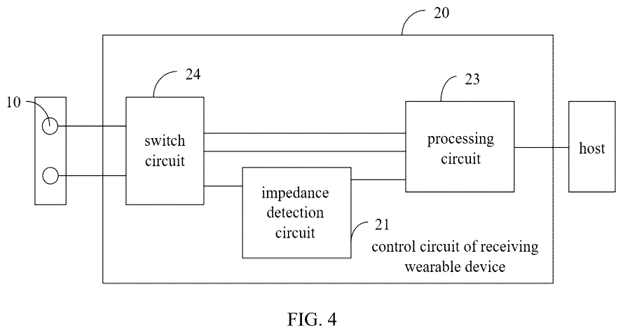

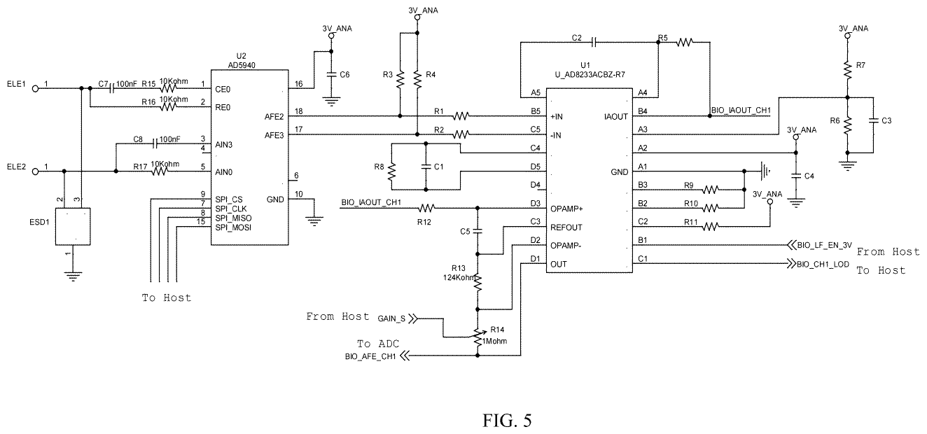

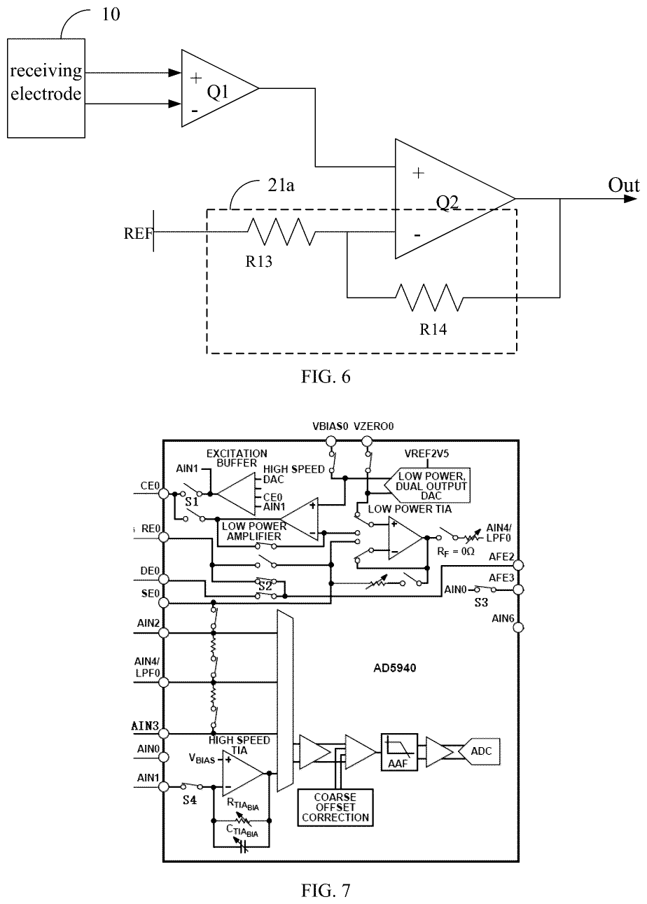

As shown in FIG. 4 to FIG. 6, in an embodiment, the control circuit of the wearable device 20 includes: an impedance detection circuit 21 and a processing circuit.

A detection end of the impedance detection circuit 21 is electrically connected to the receiving electrode 10; and the impedance detection circuit 21 is used to detect the present skin impedance of the user.

The processing circuit is electrically connected to the impedance detection circuit 21; the processing circuit is used to calculate the present skin impedance and the initial skin impedance detected by the impedance detection circuit 21 to obtain a change proportional coefficient, and adjust the gain of the excitation signal based on the change proportional coefficient.

In this embodiment, the quantity of receiving electrodes 10 is at least two. Two receiving electrodes 10, an impedance detection circuit 21 and human skin form a loop to perform impedance detection on the human skin between the two receiving electrodes 10. Or, when a part of a human body wearing a transmitting wearable device contacts a part of a human body wearing a wearable device, the transmitting electrode, the receiving electrode 10, the impedance detection circuit 21 and the human skin form a loop to perform impedance detection on the human skin between the transmitting electrode and the receiving electrode 10. Further, the impedance detection of the human skin can be automatically triggered by the control circuit of the wearable device 20. For example, since the user puts on the wearable device, the control circuit of the wearable device 20 performs a human skin impedance detection at intervals of 1s, the skin impedance detected in the first of the two adjacent detections is used as the initial skin impedance, and the skin impedance detected in the second is the present skin impedance. Or, the impedance detection of the human skin can be actively triggered by the user, for example, the user clicks on the corresponding functional area to trigger the human skin impedance detection. The impedance detection circuit 21 can be implemented using an AD5940 chip, and the processing circuit 23 can be implemented using an AD8233 chip.

The processing circuit 23 calculates the ratio of the present human body impedance detected by the impedance detection circuit 21 to the initial human body impedance, and obtains the change proportional coefficient τ of the skin impedance. The change proportional coefficient τ and the initial gain are calculated to obtain the target gain, and the gain of the excitation signal is adjusted to the target gain, so that the gain of the excitation signal always follows the change of the skin impedance, ensuring that the excitation signal output by the control circuit of the wearable device 20 can be correctly identified.

In this embodiment, the impedance detection circuit 21 detects the present skin impedance of the user, and the processing circuit 23 calculates the present skin impedance and the initial skin impedance to obtain the change proportional coefficient of the skin impedance, and calculates the change proportional coefficient and the initial gain to obtain the target gain. Therefore, the gain of the signal amplifying circuit to the excitation signal is adjusted to the target gain, so that the gain of the signal amplifying circuit to the excitation signal always follows the change of skin impedance, ensuring that the excitation signal output by the control circuit of the wearable device 20 can be correctly identified.

In an embodiment, the processing circuit 23 includes: a main control circuit, a signal amplifying circuit and a gain adjustment circuit 21a.

The main control circuit is electrically connected to the impedance detection circuit 21, and the main control circuit is used to calculate the present skin impedance and the initial skin impedance detected by the impedance detection circuit 21 to obtain a change proportional coefficient.

The input end of the signal amplifying circuit is electrically connected to the receiving electrode 10, and the signal amplifying circuit is used to amplify and output the received excitation signal.

The input end of the gain adjustment circuit 21a is connected to the main control circuit, and the output end of the gain adjustment circuit 21a is connected to the controlled end of the signal amplifying circuit.

The main control circuit is further used to control the gain adjustment circuit 21a to adjust the gain of the signal amplifying circuit based on the change proportional coefficient.

In an embodiment, the control circuit of the wearable device 20 further includes an analog-to-digital conversion circuit. The input end of the analog-to-digital conversion circuit is connected to the output end of the signal amplifying circuit, and the analog-to-digital conversion circuit is used to convert the excitation signal output by the signal amplifying circuit into a digital signal and output it so that the upper-level device can process and identify the digital signal.

In this embodiment, the main control circuit calculates the ratio of the present human body impedance detected by the impedance detection circuit 21 to the initial human body impedance, obtains the change proportional coefficient τ of the skin impedance, and calculates the change proportional coefficient τ and the initial gain to obtain the target gain, and controls the gain adjustment circuit 21a to adjust the gain of the signal amplifying circuit to the target gain. Therefore, the gain of the signal amplifying circuit to the excitation signal always follows the change of skin impedance, ensuring that the excitation signal output by the control circuit of the wearable device 20 can be correctly identified.

As shown in FIG. 5, in an embodiment, the gain adjustment circuit 21a includes: a variable resistor R14.

The input end of the variable resistor R14 is connected to the input end of the signal amplifying circuit, the output end of the variable resistor R14 is connected to the output end of the signal amplifying circuit, and the controlled end of the variable resistor R14 is connected to the main control circuit.

The main control circuit is used to adjust the resistance value of the variable resistor R14 based on the change proportional coefficient.

In this embodiment, the input end of the signal amplifying circuit includes a positive input end and an inverting input end. The positive input end of the signal amplifying circuit is used to access the reference voltage, and the inverting input end of the signal amplifying circuit is connected to the receiving electrode 10. The input end of the variable resistor R14 is connected to the inverting input end of the signal amplifying circuit, and the output end of the variable resistor R14 is connected to the output end of the signal amplifying circuit to amplify the received excitation signal. The main control circuit adjusts the resistance value of the variable resistor R14 based on the change proportional coefficient, and then adjusts the gain of the signal amplifying circuit to the excitation signal.

Furthermore, the gain adjustment circuit 21a further includes a fourth resistor R13. The input end of the fourth resistor 13 is used to access the reference voltage, and the output end of the fourth resistor 13 is connected to the input end of the signal amplifying circuit. The controlled end of the variable resistor R14 is connected to the processing circuit 23. The gain of the signal amplifying circuit is determined by the fourth resistor R13 and the variable resistor R14: G=1+R14/R13. The processing circuit 23 determines the target resistance value of R14 based on the change proportional coefficient, and adjusts the resistance value of R14 to the target resistance value through the GAIN_S pin, thereby adjusting the gain of the signal amplifying circuit. For example, the fourth resistor R13 has a fixed resistance of 124 Kohm, and the resistance value of the variable resistor R14 can be adjusted between 0˜1 Mohm, and the gain range of the signal amplifying circuit can also vary between 1˜9V/V.

After the main control circuit detects and calculates the target gain, it can quickly adjust the resistance of the variable resistor R14 based on the target gain, thereby adjusting the gain of the signal amplifying circuit to the excitation signal. When the target gain is greater than the initial gain, the resistance of the variable resistor R14 is increased, thereby increasing the gain of the signal amplifying circuit to the excitation signal. When the target gain is less than the initial gain, the resistance of the variable resistor R14 is reduced, thereby reducing the gain of the signal amplifying circuit to the excitation signal. In this embodiment, the gain of the signal amplifying circuit to the excitation signal is adjusted by adjusting the resistance of the variable resistor R14, and the gain adjustment is simple and rapid.

In an embodiment, the signal amplifying circuit includes: a first amplifier circuit and a second amplifier circuit.

The input end of the first amplifier circuit is the input end of the signal amplifying circuit, and the first amplifier circuit is used to perform a first-stage amplification on the received excitation signal.

The input end of the second amplifier circuit is connected to the output end of the first amplifier circuit, the output end of the second amplifier circuit is the output end of the signal amplifying circuit, and the controlled end of the second amplifier circuit is connected to the processing circuit 23. The second amplifier circuit is used to perform secondary amplification on the excitation signal output by the first amplifier circuit.

The processing circuit 23 is used to adjust the gain of the second amplifying circuit based on the change proportional coefficient.

In this embodiment, the first amplifier circuit includes a first amplifier Q1, and the first amplifier Q1 can be an instrument amplifier or a power amplifier. The second amplifier circuit includes a second amplifier Q2, and the second amplifier Q2 can be an instrument amplifier or a power amplifier. The gain of the first amplifier circuit is fixed, and the gain of the second amplifier circuit is adjustable. For example, the first amplifier circuit has a fixed gain of 100 V/V, and the gain of the second amplifier circuit is adjustable between 1 V/V and 9 V/V, and the gain range of the signal amplifying circuit is adjustable between 100 V/V and 900 V/V.

In this embodiment, the first amplifier circuit and the second amplifier circuit perform two-stage amplification on the received excitation signal to improve the gain of the excitation signal. By adjusting the gain of the second amplifier circuit to adjust the total gain of the signal amplifying circuit, there is no need to adjust the gain of the first amplifier circuit, and the gain adjustment is simpler and more convenient.

In an embodiment, the quantity of the receiving electrodes 10 is at least two; the impedance detection circuit 21 is further used to measure the skin impedance between the two receiving electrodes 10 to obtain the present skin impedance of the user.

In this embodiment, the impedance detection circuit 21 can complete the detection of human skin impedance through the loop formed by the two receiving electrodes 10 and the human skin. There is no need for the participation of the transmitting wearable device, and the detection is more convenient and quicker. In addition, the receiving electrode 10 is provided at the wearable device, and the distance between the two receiving electrodes 10 is fixed, and no displacement will occur to affect the impedance detection result, thereby ensuring that the change ratio coefficient obtained by calculating the present skin impedance and the initial skin impedance is more accurate.

In an embodiment, the receiving control circuit of the wearable device 20 further includes a switch circuit 24.

The controlled end of the switch circuit 24 is connected to the processing circuit 23, the input end of the switch circuit 24 is electrically connected to the receiving electrode 10, the first output end of the switch circuit 24 is connected to the detection end of the impedance detection circuit 21, and the second output end of the switch circuit 24 is connected to the input end of the signal amplifying circuit.

When performing human skin impedance detection, the processing circuit 23 controls the input end of the switch circuit 24 to be connected to the first output end. When human skin impedance detection is not performed or is completed, the processing circuit 23 controls the input end of the switch circuit 24 to be connected to the second output end.

In this embodiment, as shown in FIG. 7, the switch circuit 24 may be a switch matrix integrated in the impedance detection circuit 21. When performing human skin impedance detection, the processing circuit 23 controls the switch matrix to disconnect the signal amplifying circuit of the receiving electrode 10 and connect the receiving electrode 10 to the impedance detection circuit 21 to achieve impedance detection. When the human skin impedance detection is not performed or the human skin impedance detection is completed, the switch matrix switches the receiving electrode 10 to connect to the signal amplifying circuit to amplify and output the received excitation signal. Or, the switch circuit 24 may also be an externally connected hardware circuit, such as a triode, a Metal-Oxide-Semiconductor (MOS) tube, etc.

As shown in FIG. 5, in an embodiment, the switch circuit 24 is a switch matrix integrated in the impedance detection circuit 21. The receiving control circuit of the wearable device 20 further includes a first resistor R15, a second resistor R16, a third resistor R17, a first capacitor C7 and a second capacitor C8. There are two receiving electrodes 10, a first end of the first capacitor and an input end of the second resistor R16 are connected to one receiving electrode 10, and one end of the second capacitor and an input end of the third resistor are connected to another receiving electrode 10. AFE2 pin and AFE3 pin are connected to the input end of the signal amplifying circuit.

When performing human skin impedance detection, the processing circuit 23 controls the switch circuit 24 to disconnect the path between the REO pin and the AFE2 pin, and disconnect the path between the AIN0 pin and the AFE3 pin, so as to disconnect the path between the receiving electrode 10 and the signal amplifying circuit; and controls the switch circuit 24 to connect the CEO pin and the AIN3 pin to the impedance detection circuit 21 for impedance detection.

When human skin impedance detection is not performed or is completed, the processing circuit 23 controls the switch circuit 24 to turn on the path between the REO pin and the AFE2 pin, and the path between the AIN0 pin and the AFE3 pin, so as to connect the path between the receiving electrode 10 and the signal amplifying circuit; and controls the switch circuit 24 to disconnect the CEO pin and the AIN3 pin from the impedance detection circuit 21.

In this embodiment, the connection between the receiving electrode 10 and the impedance detection circuit 21 and the signal amplifying circuit is switched by the switching circuit 24. When performing impedance detection, the receiving electrode 10 is connected to the impedance detection circuit 21 and disconnected from the signal amplifying circuit to ensure that the impedance detection is not interfered by the excitation signal. When the human skin impedance detection is not performed or the human skin impedance detection is completed, the receiving electrode 10 is connected to the signal amplifying circuit and disconnected from the impedance detection circuit 21 to ensure that the excitation signal is not interfered by the signal of the impedance detection circuit 21 when receiving the excitation signal.

In an embodiment, the control circuit of the wearable device 20 further includes a signal sampling circuit.

The input end of the signal sampling circuit is connected to the output end of the signal amplifying circuit, and the output end of the signal sampling circuit is connected to the processing circuit 23.

The signal sampling circuit is used to collect the excitation signal output by the signal amplifying circuit.

The processing circuit 23 is further used to extract the characteristic values of multiple excitation signals collected by the signal sampling circuit within a preset time length, and calculate the average value of the multiple characteristic values to obtain the characteristic value of the present click position of the user; and determine the click position based on the characteristic value of the click position.

In this embodiment, the signal sampling circuit samples the excitation signal output by the signal amplifying circuit based on a preset sampling rate. In order to ensure the continuity of the characteristic value, the processing circuit 23 extracts the characteristic value by setting a time window and an incremental window, and calculates the average value of multiple characteristic values within a preset time length, obtains the characteristic value of the present click position of the user and outputs it to the upper-level device. The upper-level device compares and matches the characteristic value of the click position with multiple position standard characteristic values representing different click positions, and determines the position corresponding to the position standard characteristic value that best matches the characteristic value of the click position as the present click position of the user based on the matching result.

In this embodiment, the position standard characteristic values that characterize different click positions can be set before leaving the factory. Or, the amplitude of the excitation signal output by the wearable device when the user clicks on different click positions when wearing it for the first time can be collected as the position standard characteristic value. Since each person's skin condition is different, different users set the initial skin impedance and position standard characteristic value based on their own skin condition when wearing it. The initial value is collected when the user wears it for the first time. If the user is not changed later, there is no need to collect it again. If the user is changed, the initial value needs to be collected again.

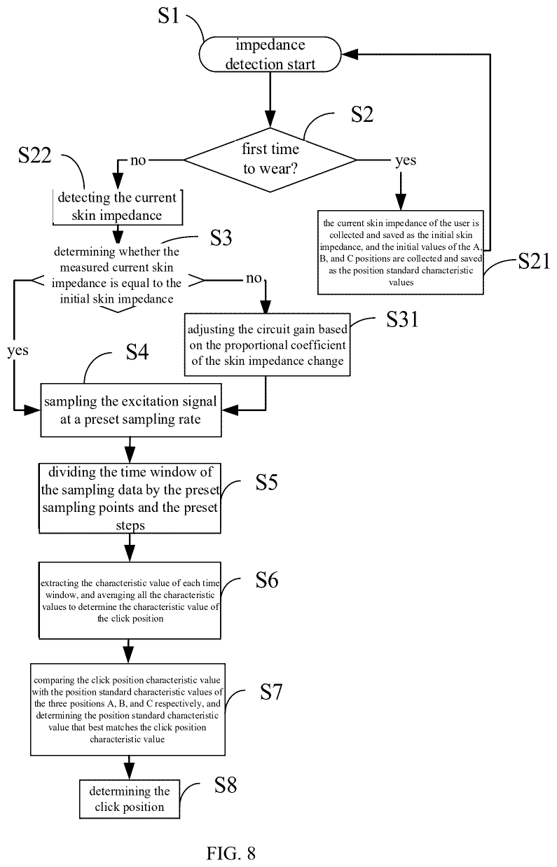

In an embodiment, as shown in FIG. 8, the signal sampling circuit samples the received excitation signal at a sampling rate of 3.2 kHz. Every 30 sampling points in the sampled data are divided into a time window, with 20 sampling points as a step, that is, in two adjacent time windows, the first time window slides 20 sampling points to become the second time window. For example, the first time window is the 1st-30th sampling points, and the second time window is the 21st-50th sampling points. There are 10 sampling points overlapping between the two time windows, and the overlapping part is the incremental window. The processing circuit 23 extracts the maximum value fMAX of the amplitude of all sampling points in each time window within the preset duration, and calculates the average value of all maximum values fMAX to obtain the click position characteristic value. The maximum value fMAX is a kind of signal characteristic value, which is the maximum value of the absolute value of all sampling points in each time window. In addition, the average absolute value can also be extracted as the characteristic value.

This embodiment extracts the characteristic value of the excitation signal and calculates its average value to obtain the characteristic value of the click position, which can represent the amplitude of the excitation signal at the present time, avoiding the characteristic value of the click position being too large or too small due to interference from signal fluctuations, thereby ensuring the accuracy of position recognition.

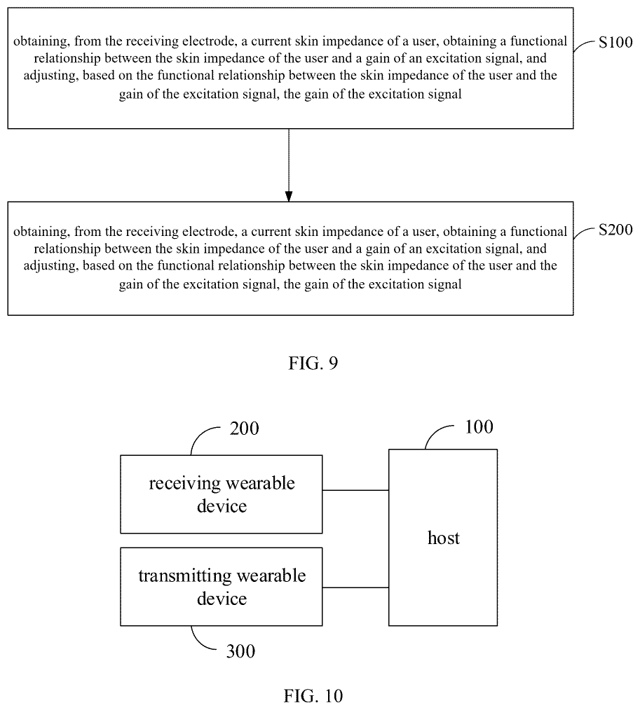

As shown in FIG. 9, the present application further provides a signal processing method, which is applied to the above-mentioned wearable device, the wearable device includes a receiving electrode; and the signal processing method includes:
S100: obtaining, from the receiving electrode, a present skin impedance of a user, obtaining a functional relationship between the skin impedance of the user and a gain of an excitation signal, and adjusting, based on the functional relationship between the skin impedance of the user and the gain of the excitation signal, the gain of the excitation signal; and,
S200: amplifying the excitation signal based on the adjusted gain to determine a present click position of the user in response to receiving the excitation signal.

In this embodiment, the transmitting wearable device has a transmitting electrode that contacts the skin of the user when the transmitting wearable device is worn by the user. The excitation signal is loaded onto the skin surface through the transmitting electrode and is conducted to the receiving electrode of the wearable device through the human skin. The receiving electrode receives the excitation signal on the skin surface, and after amplification and analog-to-digital conversion by the control circuit of the wearable device, the analog excitation signal is converted into a digital signal that can be recognized by the machine and transmitted to the upper-level device. The voltage value of the converted digital signal is compared with a plurality of position standard characteristic values representing different click positions for matching, and the position corresponding to the position standard characteristic value that best matches the excitation signal collected by the receiving electrode is determined as the present click position of the user based on the matching result.

When a click operation is required, the human body part wearing the transmitting wearable device performs a click action on the human body part wearing the wearable device. The waveform change rules of the excitation signal collected by the receiving electrode at different positions are basically the same, and all of them show click actions. Therefore, it is difficult to distinguish different click positions by action recognition alone. When clicking different positions, the resistance between the receiving electrode and the transmitting wearable device is different, the amplitude of the excitation signal waveform collected at the receiving end is different, and the amplitude of the excitation signal output to the upper-level device after amplification, analog-to-digital conversion, etc., by the control circuit of the wearable device is also different. Therefore, the present click position can be determined by comparing and matching the amplitude of the excitation signal output by the wearable device with multiple position standard characteristic values representing different click positions. The transmitting wearable device and/or the wearable device can be a watch, a bracelet or a ring, etc., which is not limited here. This embodiment is illustrated by taking a watch as an example.

For example, in an AR/VR application, a selection menu appears on a certain part of the hand wearing the wearable device (the left palm is taken as an example here), and the right hand wearing the transmitting wearable device can click on the menu to select and perform other operations. The left palm can be divided into click areas representing different functional modules. In this embodiment, three areas (A, B and C) are defined, as shown in FIG. 1A.

When the left and right hands are in contact, a conductive loop is formed between the transmitting electrode and the receiving electrode, as shown by the dotted line in FIG. 1B. When the right hand clicks on the three positions A, B, and C respectively, the resistance of the conductive loop is different due to the different path lengths. When the excitation voltage is fixed, the longer the path, the greater the resistance, the smaller the amplitude of the excitation signal received by the receiving electrode, and the smaller the amplitude of the excitation signal output after amplification, analog-to-digital conversion, etc. The shorter the path, the smaller the resistance, the greater the amplitude of the excitation signal received by the corresponding receiving electrode, and the greater the amplitude of the excitation signal output after amplification, analog-to-digital conversion, etc. In this embodiment, the resistance between the transmitting electrode-A-receiving electrode is defined as RA1, the resistance between the transmitting electrode-B-receiving electrode is defined as RB1, and the resistance between the transmitting electrode-C-receiving electrode is defined as RC1, RA1<RB1<RC1, and the corresponding collected excitation signal amplitude VA1>VB1>VC1, and the waveforms are shown in FIG. 2A and FIG. 2B. FIG. 2A and FIG. 2B show the excitation signal amplitude waveforms collected by clicking on positions A, B, and C respectively under normal skin conditions at room temperature when the excitation voltage, excitation frequency, and skin resistance remain unchanged. At this time, the present skin impedance of the user obtained by the receiving electrode is 3.2 MΩ.

However, human skin resistance will change with the skin surface. For example, when the skin surface sweats and the skin becomes moist, the skin resistance will decrease. When the skin resistance changes, the amplitudes of the excitation signals VA1, VB1, and VC1 collected by the receiving electrodes will also change.

The relationship between the amplitude of the excitation signal output by the wearable device, the skin impedance, and the gain of the excitation signal by the control circuit of the wearable device is:

$$V\_emg = ( V\_excitation \_voltage / Z ) ⋆ R\_receiving \_circuit ⋆ Gain ,$$

V_emg is the amplitude of the excitation signal output by the wearable device, that is, VA1, VA2, VA3;
V_excitation_voltage is the excitation voltage loaded by the transmitting wearable device and is a fixed value;
Z is the skin impedance between the transmitting electrode and the receiving electrode 10, i.e., RA1, RB1, RC1;
R_receiving_circuit is the resistance value of the receiving circuit part of the wearable device, which can be regarded as a fixed value; and
Gain is the gain of the control circuit of the wearable device to the excitation signal.

When the V_excitation_voltage, R_receiving_circuit and the gain Gain of the excitation signal of the control circuit of the wearable device 20 remain unchanged, the change in the amplitude V_emg of the excitation signal output by the wearable device is inversely linearly related to the change in the skin impedance Z, that is, the smaller the skin impedance, the larger the amplitude of the excitation signal output by the wearable device; and the larger the skin impedance, the smaller the amplitude of the excitation signal output by the wearable device to the upper-level device.

FIG. 3A and FIG. 3B show the waveforms of the amplitude of the excitation signal collected by clicking on the three positions A, B, and C respectively when the excitation voltage and gain remain unchanged and the skin surface is wet. At this time, the present skin impedance of the user obtained by the receiving electrode 10 is 2.2 MΩ. Comparing FIG. 2 and FIG. 3, it can be seen that the skin impedance decreases, and the amplitude of the corresponding excitation signal increases. When the excitation signal transmitted to the upper-level device is compared and matched with multiple position standard signals representing different click positions, the position standard signal representing the click position may be matched with a position standard signal different from the actual click position of the user, which leads to misjudgment.

In order to ensure the accuracy of click position recognition, it is necessary to minimize the difference between the amplitude of the excitation signal output by the wearable device when the skin impedance changes and the amplitude of the excitation signal output by the wearable device under normal skin conditions. The present application measures the present skin impedance of the user through the control circuit of the wearable device 20 to obtain the change relationship of the skin impedance. The gain of the excitation signal is adjusted based on the change rule of the skin impedance and the functional relationship between the skin impedance of the user and the gain of the excitation signal, so that the amplitude of the excitation signal output by the wearable device to the upper-level device follows the change of human skin impedance. Regardless of how the skin impedance of the user changes, it is ensured that the difference between the amplitude of the excitation signal output by the wearable device and the amplitude of the excitation signal output under normal skin conditions does not exceed the preset amplitude range.

In an embodiment, the quantity of receiving electrodes 10 is at least two, and the control circuit of the wearable device 20 measures the human skin impedance between the two receiving electrodes 10 to obtain the present skin impedance of the user. The human body is a conductor, and a small piece of skin changes in the same way as the entire human skin. Therefore, the impedance change rule of this small piece of skin can represent the impedance change rule of the entire arm skin.

Based on the functional relationship between the present skin impedance of the user and the skin impedance of the user and the gain of the excitation signal, V_emg=(V_excitation_voltage/Z)*R_receiving_circuit*Gain, it can be known that when the V_excitation_voltage and R_receiving_circuit remain unchanged, when the skin impedance Z between the transmitting electrode and the receiving electrode 10 changes, it is necessary to ensure that the amplitude difference between the excitation signal output by the wearable device and the amplitude difference of the excitation signal output under the normal skin state does not exceed the preset amplitude range. Then, when the human body impedance changes, the gain Gain of the control circuit of the wearable device 20 to the excitation signal needs to be adjusted accordingly. The skin impedance Z between the transmitting electrode and the receiving electrode 10 is in an inverse linear relationship with the gain Gain of the control circuit of the wearable device 20 to the excitation signal. When the skin impedance Z becomes larger, the gain Gain of the control circuit of the wearable device 20 to the excitation signal needs to be reduced accordingly. When the skin impedance Z becomes smaller, the gain Gain of the control circuit of the wearable device 20 to the excitation signal needs to be increased accordingly. In an embodiment, based on the functional relationship between the present skin impedance of the user and the skin impedance of the user and the gain of the excitation signal, the target gain can be determined as:

G1-G0*Z1/Z0; where G1 is the target gain, G0 is the initial gain, Z1 is the present skin impedance, and Z0 is the initial skin impedance.

The control circuit of the wearable device 20 adjusts the gain of the received excitation signal to the target gain, so as to adjust the excitation signal output to the upper-level device in accordance with the change of skin impedance when the skin impedance changes, so that the difference between the excitation signal output to the upper-level device after the skin impedance changes and the excitation signal output to the upper-level device under normal skin conditions is within the preset amplitude range, ensuring that the upper-level device can correctly identify the present click position of the user.

In this embodiment, the initial skin impedance can be set before leaving the factory. Or, the skin impedance of the user when wearing it for the first time can be collected as the initial skin impedance. Since each person's skin condition is different, different users set the initial skin impedance and position standard characteristic values based on their own skin conditions when wearing it. The initial value is collected when the user wears it for the first time. If the user is not changed later, there is no need to collect it again. If the user is changed, the initial value needs to be collected again.

The present application obtains the present skin impedance of the user, and determines the actual change of the skin impedance of the user based on the present skin impedance and the initial skin impedance of the user. based on the actual change of the skin impedance of the user, the gain of the received excitation signal of the control circuit of the wearable device is adjusted accordingly, and the excitation signal is amplified based on the adjusted gain, so that the difference between the amplitude of the excitation signal output by the wearable device and the amplitude of the excitation signal output under normal skin conditions does not exceed the preset amplitude range, avoiding the error recognition caused by the amplitude of the output excitation signal being too large or too small, and improving the accuracy of click position recognition.

In an embodiment, the functional relationship between the skin impedance of the user and the gain of the excitation signal is specifically:

$$V\_emg = ( V\_excitation \_voltage / Z ) ⋆ R\_receiving \_circuit ⋆ Gain ;$$

V_emg is an amplitude of the excitation signal output by the wearable device, V_excitation_voltage is an excitation voltage loaded by the transmitting wearable device, Z is a skin impedance between a transmitting electrode and the receiving electrode, R_receiving_circuit is a resistance of a receiving circuit in the wearable device, and Gain is a gain of a control circuit of the wearable device applied to the excitation signal.

When the V_excitation_voltage and the R_receiving_circuit remain unchanged, in order to ensure that the amplitude difference between the excitation signal output by the wearable device and the excitation signal output under normal skin conditions does not exceed the preset amplitude range, when the human body impedance changes, the gain of the control circuit of the wearable device to the excitation signal needs to be adjusted accordingly. The skin impedance Z between the transmitting electrode and the receiving electrode is in an inverse linear relationship with the gain Gain of the control circuit of the wearable device to the excitation signal. When the skin impedance Z increases, the gain Gain of the control circuit of the wearable device to the excitation signal needs to be reduced accordingly. When the skin impedance Z decreases, the gain Gain of the control circuit of the wearable device to the excitation signal needs to be increased accordingly. In this way, the amplitude of the excitation signal output to the upper-level device after the skin impedance changes always follows the change of the human body impedance, reducing the difference with the excitation signal output to the upper-level device under normal skin conditions, and ensuring that the upper-level device can correctly identify the present click position of the user.

In an embodiment, the step of the adjusting, based on the functional relationship between the skin impedance of the user and the gain of the excitation signal, the gain of the excitation signal includes:
determining, based on the functional relationship between the skin impedance of the user and the gain of the excitation signal, a target gain:
G1=G0*Z1/Z0; where G1 is the target gain, G0 is an initial gain, Z1 is the present skin impedance, and Z0 is an initial skin impedance; and
adjusting the gain of the signal amplifying circuit of the wearable device to the target gain.

The ratio of the initial skin impedance to the present skin impedance is calculated to obtain the change rule of the skin impedance: Z11/Z12=τ, where τ is the proportional coefficient of the change of the skin impedance. In connection with the above functional relationship V_emg=(V_excitation_voltage/Z)*R_receiving_circuit*100*Gain, the functional relationship between the present skin impedance and the skin impedance of the user and the gain of the excitation signal can be obtained as follows:

V_emg0/V_emg1=(Z0/Z1)*(Gain0/Gain1). V_emg0 is the amplitude of the excitation signal collected by the receiving electrode at the initial moment, V_emg1 is the amplitude of the excitation signal collected by the receiving electrode at the present moment, Gain0 is the initial gain, and Gain1 is the target gain.

To ensure that the difference between the amplitude of the excitation signal collected by the receiving electrode at the present moment and the amplitude of the excitation signal collected by the receiving electrode at the initial moment is as small as possible, in this embodiment, V_emg0 and V_emg1 can be regarded as approximately equal. That is, G1=G0*Z1/Z0, and Z11/Z12=τ is substituted to obtain the target gain: Gain2=Gain1/τ.

The control circuit of the wearable device adjusts the gain of the received excitation signal based on the target gain, so as to adjust the excitation signal output to the upper-level device based on the change of skin impedance when the skin impedance changes, so that the difference between the excitation signal output to the upper-level device after the skin impedance changes and the excitation signal output to the upper-level device under normal skin conditions is within a preset amplitude range, ensuring that the upper-level device can correctly identify the present click position of the user.

In an embodiment, the signal processing method further includes:
obtaining the present skin impedance of the user as an initial skin impedance when the user is wearing the wearable device for a first time.

In this embodiment, when the user wears the wearable device, the query information “Is it the first time to wear it?” is output in the form of images and/or audio, and the user can click the corresponding functional area (such as the left palm) to select yes or no. If the user selects yes, the wearable device measures the present skin impedance of the user as the initial skin impedance of the user. If the user selects no, the function menu option is entered.

This application will be explained below in conjunction with the accompanying drawings.

As shown in FIG. 8, in an embodiment, the signal processing method includes the following steps.
S1: starting impedance detection;
the impedance detection can be actively triggered by the user, or the impedance detection can be performed at regular intervals between the control circuits of the receiving wearable device.
S2: determining whether the user is wearing the device for the first time;

For example, when the user wears the wearable device, the query information “Is it the first time to wear it?” is output in the form of images and/or audio, and the user can click the corresponding functional area (such as the left palm) to select yes or no. If the user selects yes, the wearable device measures the present skin impedance of the user as the initial skin impedance of the user. If the user selects no, the function menu option is entered.
S21: in response to determining that the user is wearing the device for the first time, the present skin impedance of the user is collected and saved as the initial skin impedance, and the initial values of the A, B, and C positions are collected and saved as the position standard characteristic values;
S22: in response to determining that the user is not wearing the device for the first time, measuring the present skin impedance;
S3: after step S22, determining whether the measured present skin impedance is equal to the initial skin impedance;
S31: in response to that the present skin impedance is not equal to the initial skin impedance, adjusting the circuit gain based on the proportional coefficient of the skin impedance change. The specific method of adjusting the circuit gain based on the proportional coefficient of the skin impedance change can refer to steps S100 and S200, which will not be repeated here.
S4: sampling the excitation signal at a preset sampling rate (e.g., 3.2 kHz);
S5: dividing the time window of the sampling data by the preset sampling points and the preset steps.

For example, the sampling data is divided into a time window of 30 points, with a step of 30 points, that is, in two adjacent time windows, the first time window slides 20 sampling points to become the second time window. For example, the first time window is the 1st to 30th sampling points, and the second time window is the 21st to 50th sampling points. There is an overlap of 10 sampling points between the two time windows, and the overlapping part is the incremental window.

S6: extracting the characteristic value of each time window, and averaging all the characteristic values to determine the characteristic value of the click position.

For example, extracting the maximum value fMAX of the amplitude of all sampling points in each time window within the preset duration, and calculating the average value of all maximum values fMAX to obtain the click position characteristic value. The maximum value fMAX is a kind of signal characteristic value, which is the maximum value of the absolute value of all sampling points in each time window. In addition, the average absolute value can also be extracted as a characteristic value.
S7: comparing the click position characteristic value with the position standard characteristic values of the three positions A, B, and C respectively, and determining the position standard characteristic value that best matches the click position characteristic value; and
S8: determining the click position.

As shown in FIG. 10, the present application further provides a wearable system, including: a host 100; and the above-mentioned wearable device 200.

The above-mentioned wearable device 200 is electrically or wirelessly connected to the host 100; the wearable device 200 control circuit of the wearable device 200 outputs a corresponding click position signal to the host 100 based on the click position, so that the host 100 generates a corresponding image and/or audio based on the click position signal.

The detailed structure of the wearable device can refer to the above-mentioned embodiment, which will not be repeated here. It can be understood that since the above-mentioned wearable device is used in the wearable system of the present application, the embodiment of the wearable system of the present application includes all the technical solutions of all the embodiments of the above-mentioned wearable device, and the technical effects achieved are the same, which will not be repeated here.

In an embodiment, the wearable system further includes a transmitting wearable device 300.

The transmitting wearable device 300 is provided with a transmitting electrode that contacts the skin of the user when the transmitting wearable device 300 is worn by the user.

When a part of a body wearing the transmitting wearable device 300 contacts a part of the body wearing the wearable device 200, a signal channel is formed by the transmitting electrode of the transmitting wearable device 300, the human skin and the receiving electrode of the wearable device 200. The excitation signal sent by the transmitting electrode of the transmitting wearable device 300 is transmitted to the receiving electrode of the wearable device 200 through the signal channel.

In an embodiment, the excitation signal generating circuit includes: an excitation source, a filter circuit and a third amplifier circuit.

The excitation source is used to generate an excitation signal.

The input end of the filter circuit is connected to the output end of the excitation source and the filter circuit is used to filter the excitation signal.

An input end of the third amplifier circuit is connected to the output end of the filter circuit, and an output end of the third amplifier circuit is electrically connected to the transmitting electrode group. The third amplifier circuit is used to amplify and output the excitation signal output by the filter circuit.

In this embodiment, an excitation signal of a specific frequency and a specific voltage is generated by an excitation source, the excitation signal is filtered by a filtering circuit, and the excitation signal is amplified by a third amplifier circuit, so that the excitation signal can be smoothly transmitted and received. Finally, the excitation signal is loaded to the human skin through the transmitting electrode, and then transmitted to the receiving electrode in contact with the human skin through the human skin, thereby completing the transmission and reception of the excitation signal.

The above are only some embodiments of the present application, and do not limit the scope of the present application thereto. Under the inventive concept of the present application, equivalent structural transformations made based on the description and drawings of the present application, or direct/indirect application in other related technical fields are included in the scope of the present application.

What is claimed is:

1. A wearable device, comprising:
a receiving electrode; and
a control circuit of the wearable device electrically connected to the receiving electrode;
wherein when the wearable device is worn by a user, the receiving electrode is configured to contact skin of the user and receive an excitation signal sent by a transmitting wearable device through the skin of the user;
wherein the control circuit of the wearable device is configured to receive the excitation signal through the receiving electrode; and
wherein the control circuit of the wearable device is configured to:
obtain, by the receiving electrode, a present skin impedance of the user, a functional relationship between a skin impedance of the user and a gain of the excitation signal;
adjust, based on the present skin impedance and the functional relationship between the skin impedance of the user and the gain of the excitation signal, the gain of the excitation signal; and
amplify the excitation signal based on the adjusted gain to determine a present click position of the user in response to receiving the excitation signal.

2. The wearable device of claim 1, wherein the control circuit of the wearable device comprises:
an impedance detection circuit comprising a detection end electrically connected to the receiving electrode; and
a processing circuit electrically connected to the impedance detection circuit,
wherein the impedance detection circuit is configured to detect the present skin impedance of the user;
wherein the processing circuit is configured to:
calculate a change proportional coefficient based on the present skin impedance and an initial skin impedance detected by the impedance detection circuit; and
adjust the gain of the excitation signal based on the change proportional coefficient.

3. The wearable device of claim 2, wherein the processing circuit comprises:
a main control circuit electrically connected to the impedance detection circuit;
a signal amplifying circuit comprising an input end electrically connected to the receiving electrode; and
a gain adjustment circuit comprising an input end connected to the main control circuit, and an output end connected to a controlled end of the signal amplifying circuit,
wherein the main control circuit is configured to calculate the change proportional coefficient based on the present skin impedance and the initial skin impedance detected by the impedance detection circuit;
wherein the signal amplifying circuit is configured to amplify and output a received excitation signal; and
wherein the main control circuit is further configured to control the gain adjustment circuit to adjust a gain of the signal amplifying circuit based on the change proportional coefficient.

4. The wearable device of claim 3, wherein the gain adjustment circuit comprises:
a variable resistor, wherein an input end of the variable resistor is connected to an input end of the signal amplifying circuit, an output end of the variable resistor is connected to an output end of the signal amplifying circuit, and a controlled end of the variable resistor is connected to the main control circuit; and
wherein the main control circuit is configured to adjust a resistance value of the variable resistor based on the change proportional coefficient.

5. The wearable device of claim 2, comprising: at least two receiving electrodes,
wherein the impedance detection circuit is configured to measure a skin impedance between the two receiving electrodes to obtain the present skin impedance of the user.

6. The wearable device of claim 2, wherein the control circuit of the wearable device further comprises:
a switch circuit, wherein a controlled end of the switch circuit is connected to the processing circuit, an input end of the switch circuit is electrically connected to the receiving electrode, a first output end of the switch circuit is connected to a detection end of the impedance detection circuit, and a second output end of the switch circuit is connected to an input end of the signal amplifying circuit;
wherein when human skin impedance detection is performed, the processing circuit is configured to control the input end of the switch circuit to connect to the first output end of the switch circuit; and
wherein when human skin impedance detection is not performed or is completed, the processing circuit is configured to control the input end of the switch circuit to connect to the second output end of the switch circuit.

7. The wearable device of claim 2, wherein the control circuit of the wearable device further comprises: a signal sampling circuit,
wherein an input end of the signal sampling circuit is connected to an output end of the signal amplifying circuit and an output end of the signal sampling circuit is connected to the processing circuit;
wherein the signal sampling circuit is configured to collect the excitation signal output by the signal amplifying circuit; and
wherein the processing circuit is further configured to:
extract characteristic values of a plurality of excitation signals collected by the signal sampling circuit within a preset time interval;
calculate an average of the plurality of characteristic values of the plurality of excitation signals to determine characteristic values of a present click position of the user; and
determine the click position based on the characteristic values of the click position.

8. A signal processing method, applied to the wearable device of claim 1, wherein the wearable device comprises a receiving electrode, the method comprising:
obtaining, from the receiving electrode, a present skin impedance of a user;
obtaining a functional relationship between the skin impedance of the user and a gain of an excitation signal;
adjusting, based on the functional relationship between the skin impedance of the user and the gain of the excitation signal, the gain of the excitation signal; and
amplifying the excitation signal based on the adjusted gain to determine a present click position of the user in response to receiving the excitation signal.

9. The signal processing method of claim 8, wherein the functional relationship between the skin impedance of the user and the gain of the excitation signal is:
$$V\_emg = ( V\_excitation \_voltage / Z ) ⋆ R\_receiving \_circuit ⋆ Gain ;$$
wherein V_emg is an amplitude of the excitation signal output by the wearable device, V_excitation_voltage is an excitation voltage loaded by the transmitting wearable device, Z is a skin impedance between a transmitting electrode and the receiving electrode, R_receiving_circuit is a resistance of a receiving circuit in the wearable device, and Gain is a gain of a control circuit of the wearable device applied to the excitation signal.

10. The signal processing method of claim 8, wherein the adjusting, based on the functional relationship between the skin impedance of the user and the gain of the excitation signal, the gain of the excitation signal comprises:
determining, based on the functional relationship between the skin impedance of the user and the gain of the excitation signal, a target gain:
G1=G0*Z1/Z0; wherein G1 is the target gain, G0 is an initial gain, Z1 is the present skin impedance, and Z0 is an initial skin impedance; and
adjusting the gain of the signal amplifying circuit of the wearable device to the target gain.

11. The signal processing method of claim 8, further comprising:
obtaining the present skin impedance of the user as an initial skin impedance when the user is wearing the wearable device for a first time.

12. The signal processing method of claim 8, further comprising:
starting operating an impedance detection; and
determining whether the user is wearing the wearable device for a first time.

13. The signal processing method of claim 12, wherein the determining whether the user is wearing the wearable device for the first time comprises:
in response to determining that the user is wearing the wearable device for the first time, collecting the present skin impedance of the user, and collecting and saving initial values of a first position, a second position, and a third position as position standard characteristic values; and
in response to determining that the user is not wearing the wearable device for the first time, measuring the present skin impedance.

14. The signal processing method of claim 13, wherein after the in response to determining that the user is not wearing the wearable device for the first time, measuring the present skin impedance, the method further comprises:
determining whether the measured present skin impedance is equal to an initial skin impedance.

15. The signal processing method of claim 14, further comprising:
in response to determining that the present skin impedance is not equal to the initial skin impedance, adjusting a gain of a control circuit of the wearable device based on a change proportional coefficient of the skin impedance.

16. The signal processing method of claim 12, further comprising:
sampling the excitation signal at a preset sampling rate;
dividing a time window of sampling data by a preset sampling point and a preset step; and
extracting a characteristic value of each time window, and averaging all the characteristic values to determine a characteristic value of a click position;
comparing the characteristic value of the click position with the position standard characteristic values of the first position, second position, and third position respectively, and determining a position standard characteristic value that best matches the characteristic value of the click position; and
determining the click position.

17. A wearable system, comprising:
a host; and
the wearable device of claim 1,
wherein the wearable device is electrically or wirelessly connected to the host; and
wherein the control circuit of the wearable device is configured to:
output, based on the click position, a corresponding click position signal to the host to cause the host to generate a corresponding image and/or audio based on the click position signal.

18. The wearable system of claim 17, further comprising:
a transmitting wearable device provided with a transmitting electrode configured to contact a skin of a user when the transmitting wearable device is worn by the user,
wherein when a part of a body wearing the transmitting wearable device contacts a part of the body wearing the wearable device, a signal channel is formed by a transmitting electrode of the transmitting wearable device, human skin, and a receiving electrode of the wearable device; and
wherein an excitation signal sent by the transmitting electrode of the transmitting wearable device is transmitted to the receiving electrode of the wearable device through the signal channel.

19. The wearable system of claim 17, wherein the control circuit of the wearable device comprises:
an impedance detection circuit comprising a detection end electrically connected to the receiving electrode; and
a processing circuit electrically connected to the impedance detection circuit,
wherein the impedance detection circuit is configured to detect the present skin impedance of the user;
wherein the processing circuit is configured to:
calculate a change proportional coefficient based on the present skin impedance and an initial skin impedance detected by the impedance detection circuit; and
adjust the gain of the excitation signal based on the change proportional coefficient.

20. The wearable system of claim 19, wherein the processing circuit comprises:
a main control circuit electrically connected to the impedance detection circuit;
a signal amplifying circuit comprising an input end electrically connected to the receiving electrode; and
a gain adjustment circuit comprising an input end connected to the main control circuit, and an output end connected to a controlled end of the signal amplifying circuit,
wherein the main control circuit is configured to calculate the change proportional coefficient based on the present skin impedance and the initial skin impedance detected by the impedance detection circuit;
wherein the signal amplifying circuit is configured to amplify and output a received excitation signal; and
wherein the main control circuit is further configured to control the gain adjustment circuit to adjust a gain of the signal amplifying circuit based on the change proportional coefficient.
